(19) United States
(12) Patent Application Publication
(10) Pub. No.: US 2014/0072598 A1
(43) Pub. Date: Mar. 13, 2014

(54) MEDICINAL PREPARATION "RENESSANS" HAVING AN ANTIBACTERIAL, ANTI-ULCEROUS AND IMMUNO-MODULATING ACTION

(71) Applicants: Shokan Sabirkhanovich Begaliev, Almaty (KZ); Kulshat Magzumovna Nugerbekova, Astana (KZ)

(72) Inventors: Shokan Sabirkhanovich Begaliev, Almaty (KZ); Kulshat Magzumovna Nugerbekova, Astana (KZ)

(21) Appl. No.: 14/117,857

(22) PCT Filed: Nov. 10, 2011
(86) PCT No.: PCT/KZ2011/000018

§ 371 Date: Nov. 15, 2013

(30) Foreign Application Priority Data
May 16, 2011 (KZ) 2011/0501.1

(51) Int. Cl. A01N 59/12; A61K 33/18

(52) U.S. Cl. CPC A01N 59/12; A61K 33/18
USPC 424/278.1; 424/670

(57) ABSTRACT

The invention relates to the field of medicine, particularly to medical preparations of anti-bacterial, anti-ulcer and immune simulating effects, which can be used in injection form. The medical preparation “RENESSANS” contains components in the following ratio (wt %):

| | |
|---|---|
| Iodine | 0.4-2.0 |
| Potassium Iodide | 0.8-4.0 |
| Starch | 10.0-40.0 |
| Ascorbic acid | 0.4-2.0 |
| Glucose | 1.2-4.8 |
| Sodium chloride | 0.3-1.8 |
| Purified water | all the rest |

A pharmaceutically effective amount of the medical preparation is administered intravenously, intramuscularly, or orally.

### CROSS-REFERENCE TO RELATED APPLICATION

This application claims the benefit of the priority filing date of PCT application no. PCT/KZ2011/000018 filed on Nov. 10, 2011 and published in WO2012/158002 on Nov. 22, 2012. The earliest priority filing date claimed is May 16, 2011.

### FEDERALLY SPONSORED RESEARCH

Not Applicable

### SEQUENCE LISTING OR PROGRAM

Not Applicable

### BACKGROUND

The invention relates to the pharmaceutical industry, in particular, iodine-containing pharmaceuticals.

Nowadays iodine medications occupy an important place among modern antiseptics. Many infections are caused by agents that are sensitive to iodine-containing medications which exhibit fungicidal, cellulicidal and antiprotozoal actions, and which act against some spores while not developing an acquired resistance to them.

The problem of using iodine is that high concentrations are toxic, irritating the skin and mucous membranes. However, in combination with polysaccharides and polymers, iodine largely loses toxicity, and operates more slowly and continuously (during 5 hours), while maintaining anti-virus and anti-microbial properties (Nikulin V., V. Gerasimenko, 2008; E. Svirskaya, 2008).

Iodine also exhibits fungicidal, cellulicidal and antiprotozoal actions, and exhibits some activity against spores. During resorptive action, Iodine is actively involved in the metabolic processes of the body: increasing the lecithin-cholesterol ratio, lipoproteinaz (lipoprotein), and fibrinolytic activity of blood; slowing blood clotting; and lowering cholesterol and β-lipoproteins.

Iodine complexes with polymers (iodinolum, iodonatum, iodovidonum, povidon-iodine, iodopiron, iodoformium, hiniofon) are used as an antiseptic for ablution of tonsils in the fields of chronic tonsillitis, purulent otitis media, sinusitis, keratitis, abrasions, perirodontite, conjunctivitis, for the treatment of wounds, and in operations.

Povidon-iodine (iodine in the form of complex binding with iodoformium-polyvinylpyrrolidone) is known to have a wider spectrum of antibacterial actions than other known antiseptics. It manifests actions against many kinds of viruses, including HIV, in suspension experiments in vitro [Kawana R. et. al., Inactivation of human viruses by povidon-iodine in comparison with other antiseptics.—Dermatology, 1997, 195, Application No 2, pages 29-35]. Anti-infective pharmaceuticals containing povidon-iodine can be used to combat against diseases caused by HIV, chlamydia, gonococcus, treponema and herpes simplex virus [Tsutomu S., Junko S. Anti-infective against STD relating, for example, to HIV, chlamydia, gonococcus, treponema palladium or herpes simplex and device used to preserve/mix principal ingredient and base of such anti-infective—EP 0823996A1, A 01N 59/12, A 51K 9/00, applic. 15.08.96, publ. 18.02.98 and Shanbrom E. Antiviral, spermicidal vaginal gel and foam containing low molecular weight povidoneiodine. U.S. Pat. No. 5,545,401, A 61K 31/79, A 61K 33/18, applic. 02.06.94, publ. 13.08.96].

Iodomidolum is known as a medical preparation that has antiviral and antibacterial properties [patent number of RK 6730, applic. 26.01.96, publ. Nov. 16, 1998], which contains iodine, potassium iodide and sodium, a synthetic water-soluble polymer having a role in the polymer matrix, a mixture of natural polymers, such as poly-, mono- and oligosaccharides, as well as water in the following ratio, g/L: Iodine 6-10, potassium iodide or sodium 9-15, synthetic water-soluble polymer 2-4, mono-, oligo- and polysaccharides 8-120, water and rest. This medical prepartion has relatively high toxicity and is intended for the treatment of viral and bacterial diseases of other animals.

Also known are therapeutic medical preparations that have bactericidal and viricidal activity, and that contain iodine, potassium iodide and sodium, a synthetic water-soluble polymer, natural polymers such as polysaccharides, in the following ratio, g/I: 6.10 iodine, potassium iodide or sodium 9-15, a synthetic water-soluble polymer 2-4, natural polymers (polysaccharides) and mono- and oligosaccharides 8-120, water and rest (CZ 5435, A 61K 33/18, A 61K 31/715).

The disadvantage of this medical preparation is its relatively high toxicity. To influence viruses that have already penetrated cells is difficult because the cell itself protects them from impact. The adapted virus acts destructively in the cell. The challenge lies in suppressing the specific processes of biological synthesis of viral particles at the molecular level without affecting the life activity of uninfected cells.

The closest technical substance is a medical preparation, balsam “Vozrozhdenie” (provisional patent of RK under the number 13765, cl. A61K 33/18, from 10.01.2003). Balsam “Vozrozhdenie” contains (wt %) 0.5-1.5 iodine, 1.5-1.7 potassium iodide, 14.0-16.0 starch, 2.0-3.0 glucose, 8.0-10glycerol, 2.0-3.0 lactic acid, 0.8-1.0 sodium chloride, 2.0-3.0 apple cider vinegar and, the rest, distilled water. However balsam “Vozrozhdenie” is a dietary supplement, that does not detect bactericidal activity towards micobacterium and exhibits low bactericidal activity against spores, spore-forming bacteria and fungi. Balsam is designed for oral use only, and excludes introduction of a pharmaceutical composition to people by intravenous or intramuscular injection, which in turn limits the range of usage.

### SUMMARY

The goal of the invention is to provide a medical preparation with antibacterial, anti-ulcer and immune simulating properties having low toxicity, with the ability to use in injection form, and also provide a method of making the preparation. The technical result of the invention is an expansion of the assortment of medical preparations having antibacterial, anti-ulcer and immune simulating properties of low toxicity with the possibility of using the preparations in the form of an injection.

The technical result is achieved by a medical preparation containing iodine, potassium iodide, starch, ascorbic acid, glucose, sodium chloride and purified water in the following ratio (wt %):

| | |
|---|---|
| Iodine | 0.4-2.0 |
| Potassium Iodide | 0.8-4.0 |
| Starch | 10.0-40.0 |
| Ascorbic acid | 0.4-2.0 |
| Glucose | 1.2-4.8 |
| Sodium chloride | 0.3-1.8 |
| Purified water | all the rest |

### DESCRIPTION OF THE INVENTION

A change in the composition of components, namely, iodine, potassium iodide, starch, ascorbic acid, glucose and sodium chloride in aqueous solution, was unexpectedly found to provide a medical preparation suitable for use in injection form. It contains fewer components (i.e., six), relative to the prototype, which includes 9 components. The distinctive features of the claimed composition, in contrast to the prototype, lie in the composition and concentration of the components. The optimal composition of the structure that forms a balanced system of components that are stable in an aqueous solution is one that does not contain two acid components (lactic acid and apple cider vinegar), triatomic spirat, and glycerin, but contains the following (in wt. %): iodine—0.4-2.0; potassium iodide—0.8-4.0; starch—10.0-40.0; ascorbic acid—0.4-2.0; glucose—1.2-4.8; sodium chloride—0.3-1.8.

Modern principles of combinatorial chemistry were applied for the formation of the medical preparation as an integral substance. One of the main functions of the components used in the medical preparation composition is the formation of a special structure that keeps the oxidizing power of iodine in a state that exploits the transportation opportunities of particles over long distances with distribution to the whole organism.

One of the main functions of component used in this preparation is to form a special structure that keeps the oxidizing power of iodine in the state exploiting the opportunities of transportation of these particles over long distances with distribution to the whole organism. The formation of this system provides optimum composition in the following ratio: iodine—0.4-2.0; potassium iodide—0.8-4.0; starch—10.0-40.0; ascorbic acid—0.4-2.0; glucose—1.2-4.8; sodium chloride—0.3-1.8, purified water—all the rest.

A specified composition and definite concentration of components in an aqueous form forms a iodine-polymer complex that serves as the matrix and forming associates from which the active form of iodine is released. Starch and glucose are involved in the formation of the matrix and its forming associates, namely, poly- and monosaccharides with different functional groups. Introduction of potassium iodide into the medical preparation allows a physical and chemical system to form over a long period of time, preventing deactivation of the main active agent. Carbohydrates are known as essential components of bacterial cell walls, which serves as first contact with the host's immune system. Because of this fact, the mechanisms of innate immunity includes a step for identifying bacterial carbohydrates and glycoconjugates associated with the function of a number of cellular receptors, such as lectin, which were recently observed on the surface of phagocytic cells.

When drawing starch with water, even with hot water, starch granules swell and only a small part of polysaccharides starch turns to solution. Colloidal solution of starch (starch paste) is prepared by stirring in a little water and gradually pouring boiling water while stirring vigorously.

According to the generally accepted view, the formation of amylose-iodine complex is associated with a helical conformation of amylose chains, where there is a cavity channel formation in the center of the spiral with a size sufficient to accommodate iodine molecules. Starch-iodine complex forms hexagonal crystals. Formation of the iodine complex was found to not change the viscosity of amylose solutions.

A study of the starch-iodine complex structure revealed that there was zero connection between donor-acceptory link and valency, determined by the ratio of one iodine atom to eight monomer units of the polymer with a charge of iodine component. In the presence of starch, molecules J2 break down with formation of complex ion J3 and JO, and only a complex anion J3 takes part in the formation of the iodide-starch complex, which disappears from the solution. On the contrary, complex anion JO, remains in the solution and does not enter the complex. Its concentration increases sharply due to a splitting of J2.

A study of the iodine-starch reaction provides a basis to present the mechanism in the following form:

2J₂+H₂O⁺starch→HJ+HJO+starch→HJO+iodine-starch

Medical preparation components provide selective oxidation of essential molecular components of viruses and bacteria mainly by the active agent iodine.

Thus, on the one hand, the components of the claimed preparation seek to maintain its stability and structure, and on the other hand, are responsible for the direct manifestation of two basic properties (i.e., antimicrobial and immune simulating) as part of the proposed preparation that optimally matches the components of the antimicrobial character and the immune simulating action.

Together, the essential features of the medical preparation “RENESSANS” ensure active circulation of the medical preparation in the body and allows use in injection form.

The preparation method of the medical preparation “RENESSANS” is done in the following manner: prepare a solution of crystalline iodine and potassium iodide, sodium chloride and ascorbic acid in distilled water, as a suspension of starch in water; and mix all solutions to make up to 1 liter of water.

Facts Supporting the Possibility of Realization of Invention.

An exemplary method of obtaining the medical preparation: 10 g crystal iodine ground and 16 g of potassium iodide are dissolved in a small amount of purified water. To make slurry purified water, a solution of sodium chloride (9.0 g) and ascorbic acid (10.0 g) are prepared and added to 150 g of starch, and all are mixed to make up to one liter of purified water. The mixed medical preparation has a syrupy liquid form, from black to dark blue with a characteristic odor of iodine.

Examples that support properties and the ability of the medical preparation “RENESSANS”.

To investigate the safety of the medical preparation “RENESSANS,” the preparation was administered to animals intraperitoneally at various doses, undiluted and diluted in a saline solution. The selection of the dose was based on recommendations for the maximum allowable amount of fluid for injection into the peritoneal cavity, which for rats is 5 ml. The choice of injection dose was based on the classification of chemical toxicity in accordance with GOST 12.1.007-76. The medical preparation was administered intraperitoneally once daily.

During the experiment, rats were divided into control and experimental groups. There were four experimental groups at different doses and timing of administration.

The medical preparation was administered intraperitoneally to animals with breeding in saline solution of sodium chloride, and undiluted.

1. The animals of the first group were injected intraperitoneally daily to 0.06 ml of the medical preparation 3.0 ml of physiological solution that exceeds recommended therapeutic dose 1.7 times. Rats were sacrificed after 5, 10 and 15 days after the beginning of the experiment.
2. The animals of the second group were injected once by 0.66 ml of the medical preparation and 3.0 ml of saline solution, which is more than the recommended therapeutic dose 11 times, the slaughter of animals was produced in 5 days after the start of administration.
3. The animals of the third group were injected once by 2 ml of preparation and 3.0 ml of physiological solution, which is more than the recommended therapeutic dose 33 times and the slaughter of animals was produced within 10 days after the beginning of the experiment.
4. The fourth group of animals was injected once by 5 ml of pure medical preparation without dilution, which exceeds the recommended therapeutic dose 83 times and the slaughter of animals was produced within 15 days after the beginning of the experiment.

Basic data of the morphological composition analysis of the blood and leukocytic formula are shown in Table 1.

As can be seen from Table 1, the investigated preparation “RENESSANS” did not cause statistically significant changes in the blood cells of experimental animals, in comparison to control rats; therefore, the investigated medical preparation has no toxic effects on the blood system. Histological examinations of animals of control and experimental groups showed that the medical preparation “RENESSANS” did not cause changes in the histological structure of the internal organs of experimental animals, which also confirms the non-toxicity of the medical preparation.

Investigation of antimicrobial action of medical preparation was tested by in vitro method on a two-fold serial dilution of liquid media. Iodide starch and iodineamylose solutions were prepared under the action of a calculated amount of potassium poliyiodine in 0.2-1% solution of polysaccharides.

Concentration of molecular iodine in solutions ranged from 0.27 to 1%, the concentration iodinepolisaharide was about 1%.

In order to study the antibacterial effect of the medical preparation, the initial solutions were diluted with saline solution of sodium chloride. Suspension bacteria on a saline solution in an amount of 5 million cells, prepared on a daily agar culture test strain were added to each dilution of iodinepolisaharide. After a 15-minute exposure of each dilution of the medical preparation seeding was produced by a plain agar in Petri dishes. The results were determined in a day by the absence of bacterial growth, suggesting full bactericidal activity in high iodine polymer solution. The results are presented in Table 2.

Studies have been conducted to study the anti-ulcer effectiveness of medical preparation “RENESSANS” on 10 patients with gastric ulcer and dodecadactylon over 2 weeks, which showed the medical preparation's high anti-ulcer effect. Thus, during monotherapy by “RENESSANS,” ulcer healing was observed in 9 cases, regardless of their location (4—gastric ulcer: mediogastrade and antral and 5 DNA ulcers). Taking the medical preparation led to increased pain (DNA ulcer) only in one case which required an injection of anti-inflammatory medical preparation, with complete healing of ulcers of all 9 patients set during control esophagogastroscopy, and pain relief observed in 1-2-1 day of taking the medical preparation. In all cases no correlation was found between the healing effect of the medical preparation and the size of ulcers, age and sex of patients. These data show a high antiulcer efficacy of “RENESSANS” and both are associated with its reparative properties. 13 volunteers who received the medical preparation for 27 days, twice were assessed on immune status (prior to appointment and after 27 days of treatment). Studies have been done on the basis of the Laboratory of Immunology Medical Center “Sunkar”, licensed by the Almaty City Department of Health to perform clinical and immunological studies.

Evaluation of immune status includes: identification of the total number of leukocytes in peripheral (venous) blood, lymphocytes, leykoformula, relative and absolute content of major subpopulations of lymphocytes (total T lymphocytes, their helper and suppressor subsets of B lymphocytes, natural killer cells), non-specific functional activity of T lymphocyte, levels of the main classes serum immunoglobulins and circulating immune complexes, phagocytosis and functional-metabolic activity of neutrophils). The main lymphocyte subpopulations identified by commercial kits monoclonal antibodies to clusters CB3+, CB4+, CB8+−, CB16+, CB72+(Russian-made) by indirect immunofluorescence microscope “LUMAM I-3”: non-specific functional activity of T cells was studied by direct reaction of inhibition of leukocyte migration (RTML) for cell stimulation with PHA-P; concentration of the major classes of serum immunoglobulins (M, G, A) was identified by the traditional method of immunodiffusion in agar, using commercial sets of monospecific antiimmunogloouline serum: concentration of total IgE—ELISA: circulating immune complexes—with precipitation reaction in PEG-6000, followed by spectrophotometry precipitation: phagocytic activity of neutrophils was studied in reaction of phagocytosis to latex particles and testing of nitroblue tetrazolium recovery (NBT test), while a parallel formulation of both spontaneous and stimulated pirogenalom, versions of both tests were pursued (Table 3).

Thus, the proposed medical preparation “RENESSANS” can be used both for internal and injection use. The antimicrobial effect was found that it is not only comparable with aqueous solutions of iodine, but in some cases exceeds them. Results of the evaluation of the immune status during testing of the medical preparation showed an existence of immune correcting effect.

Optimum dosage forms of the medical preparation “RENESSANS” are suspension to oral use and suspension in the form of injection. The preparation of “RENESSANS” is nontoxic, has antimicrobial activity, and anti-ulcer effect and immune correcting properties.

Thus, as the result of the research the compositions of high iodine polymer with code-name “RENESSANS” were developed. Given the wide range of activity of high iodine polymer pharmaceutics, we developed the technology of producing the medical preparation “RENESSANS” in two dosage forms—suspensions for internal use and a suspension for injection. A flow sheet of producing the medical preparation “RENESSANS” in the form of suspensions for internal use and a suspension for injection are shown in FIGS. 1 and 2.

Research on the standardization and definition of shelf life.

The Preparation of “RENESSANS”, a Suspension for Oral Use.

Description. Syrupy liquid from dark blue to black in color, with a distinctive taste and aroma characteristic of iodine.

Authenticity. 5 ml of 0.1 M sodium thiosulfate was added to 5 ml of medical preparation; solution instantly decolorizes (iodine).

Ph. From 3.0 to 5.0 (potentiometric, GF XI, Issue 1, p. 113).

The dry residue. Not less than 9.0% (GF XI, Issue 2, page 148).

Density. 1.03-1.08 g/cm3 (GF XI, issue 1, page 24).

Microbiological purity. Not allowed to have a family of bacteria Enterobacteliaceae, Pseudomonas aeruginosa and Staphylococcus aureus.

The Preparation of “RENESSANS”, a Suspension for Injection.

Description. Syrupy liquid from dark blue to black in color, with a distinctive taste and aroma characteristic of iodine.

Authenticity. 5 ml of 0.1 M sodium thiosulfate was added to 5 ml of medical preparation; solution instantly decolorizes (iodine).

Ph. Value From 3.0 to 5.0 (potentiometric, GF XI, Issue 1, p. 113).

The dry residue. Not less than 9.0% (GF XI, Issue 2, page 148).

Density. 1.03-1.08 g/cm3 (GF XI, issue 1, page 24).

Sterility. The preparation is sterile. The tests are made by direct seeding in accordance with GF XI, Issue 2, page 187.

Pyrogenicity. Medical preparation is apyrogenic. A test dose of 0.1 mg diluted in 1 ml of sterile water for 1 kg of body weight intravenously (GF XI, Issue 2, p. 183).

Toxicity. The preparation is non-toxic. A test dose of 0.1 mg, dissolved in 0.5 ml of water for injection, intravenous (GF XI, Issue 2, p. 182). Time of observation is 48 hours.

Stability testing of the medical preparation “RENESSANS”, suspensions for internal use, in accordance with the Guidelines CMP/ICH/2736/99 “Stability Testing of New Medical preparation s and Preparations”.

The test results for the internal stability of the suspension of the medical preparation “RENESSANS” and a suspension for injecting the medical preparation “RENESSANS” are given in Tables 1 and 2, respectively.

Thus, the results of research on the standardization of the medical preparation “RENESSANS” on the physical and chemical parameters of suspensions for internal use and suspension in injection form showed that the developed medical preparation corresponds to regulatory requirements.

The medical preparation's expiration term date for intravenous and injection use are defined by all the parameters of quality and quantity, the expiration date for both medical preparation are set in 2 years.

The study of specific biological activity and safety of the medical preparation “RENESSANS”. Investigation of the medical preparation's antimicrobial activity was carried out by in vitro methods using a two-fold serial dilution in liquid media.

Starch iodide and iodinemilose solutions are prepared by a calculated amount of potassium poliiodide at 0.2-1% solution of polysaccharides. Concentration of molecular iodine in solutions ranged from 0.27 to 1%. The concentration of iodinepolisaharide (iodine polyvinyl alcohol) was about 1%. To study the antibacterial properties of the medical preparation, stock solutions were diluted with saline solution of sodium chloride. Iodinepolisaharide suspension of bacteria was added to each dilution in saline at 5 million cells, prepared on a daily agar culture test strain.

After a 15-minute exposure of each dilution of the medical preparation, seeding was produced on plain agar in Petri dishes. The next day the results showed an absence of bacterial growth suggesting about full bactericidal activity of the medical preparation solution “RENESSANS”.

The results are presented in Table 1.

TABLE 1
Antibacterial spectrum of iodinepolisaharide

| | Iodine concentration at which microbial | | |
|---|---|---|---|
| | growth is fully absent, y/ml | | |
| Test-microbe | Iodine starch | Iodinemilose | “RENESSANS” |
| Staph. aureus 209-p | 3-10 | 3-5.4 | 2-3.6 |
| Bac. subtilis 6633 | 5-10 | 3.7 | 1.4-2.7 |
| ATCC | | | |
| Esch. coli 25922 | 10 | 2.4 | 1.6 |
| ATCC | | | |
| Proteus vulgaris | 10-20 | 6.7 | 4.6 |
| 6896 ATCC | | | |
| Ps. aeroginosa | 10 | 5.8 | 2.7 |
| 27853 ATCC | | | |
| Microsporum canis | 20 | 6.5 | 3.4 |
| 3/84 | | | |
| Trichophyton | 10 | 10 | 10 |
| gypscum 5.85 | | | |
| Candida albicaps | 10 | 10 | 10 |
| 1755 | | | |

Studies conducted on the anti-ulcer effectiveness of the medical preparation “RENESSANS” on 10 patients with gastric ulcer and dodecadactylon over 2 weeks showed a high anti-ulcer effect of the medical preparation. Thus, during monotherapy by “RENESSANS,” ulcer healing was observed in 9 cases, regardless of their location (4—gastric ulcer: mediogastrade and antral and 5 DNA ulcers). Only in one case of taking the medical preparation did increased pain (DNA ulcer) result, thereby warranting an injection of anti-inflammatory medical preparation. During control esophagogastroscopy all 9 patients, complete healing of ulcers occurred, and pain relief was observed on 1-2-1 days of taking the medical preparation. In all cases, no correlation was found between the healing effect of the medical preparation and the size of ulcers, age and sex of patients. This data shows a high antiulcer efficacy of “RENESSANS” and both studies are associated with its reparative properties. 13 volunteers who received the medical preparation for 27 days, twice were assessed on the immune status (prior to appointment and after 27 days of treatment). Studies have been done on the basis of the Laboratory of Immunology Medical Center “Sunkar”, licensed by the Almaty City Department of Health to perform clinical and immunological studies.

Evaluation of immune status includes: the identification of the total number of leukocytes in peripheral (venous) blood, lymphocytes, leykoformula, relative and absolute content of major subpopulations of lymphocytes (total T lymphocytes, their helper and suppressor subsets of B lymphocytes, natural killer cells), non-specific functional activity of T lymphocyte, levels of the main classes serum immunoglobulins and circulating immune complexes, phagocytosis and functional-metabolic activity of neutrophils. The main lymphocyte subpopulations identified by commercial kits, monoclonal antibodies to clusters CB3+, CB4+, CB8+−, CB16+, CB72+(Russian-made) by indirect immunofluorescence microscope “LUMAM 1-3,” non-specific functional activity of T cells, were studied by direct reaction of inhibition of leukocyte migration (RTML) for cell stimulation with PHA-P. Concentration of the major classes of serum immunoglobulins (M, G, A) was identified by the traditional method of immunodiffusion in agar, using commercial sets of monospecific antiimmunogloouline serum, concentration of total IgE—ELISA, circulating immune complexes—with precipitation reaction in PEG-6000, followed by spectrophotometry precipitation. Phagocytic activity of neutrophils was studied in a reaction of phagocytosis to latex particles and testing of nitroblue tetrazolium recovery (NBT test), while a parallel formulation of both spontaneous and stimulated pirogenalom, versions of both tests, were pursued. In trying to determine the significant effects of the medical preparation on the immune reactivity of organism by average values, we have not found a statistically significant effect (Table 2).

TABLE 2
The average annual value of the studied immunological
parameters during the study

| | |
|---|---|
| Immunological | Period of research |

| characteristics | Before | After | P |
|---|---|---|---|
| CD3⁺ (%) | 49.2 ± 1.7 | 51.6 ± 1.1 | >0.05 |
| CD4⁺(%) | 33.5 ± 0.7 | 35.6 ± 0.7 | >0.05 |
| CD8⁺ (%) | 21:6 ± 1.3 | 22.7 ± 0.8 | >0.05 |
| CD16⁺(%) | 14.1 ± 1.5 | 15.9 ± 1.2 | >0.05 |
| CD72⁺ (%) | 16.5 ± 1.5 | 17.1 ± 1.3 | >0.05 |
| CD4⁺CD8⁺ | 1.62 ± 0.11 | 1.58 ± 0.11 | >0.05 |
| ITML | 0.63 ± 0.03 | 0.61 ± 0.13 | >0.05 |
| Ig M (g/l) | 0.63 ± 0.08 | 0.98 ± 0.07 | <0.05 |
| Ig G (g/l) | 9.19 ± 0.83 | 9.66 ± 0.66 | >0.05 |
| Ig A (g/l) | 1.48 ± 0.20 | 1.57 ± 0.29 | >0.05 |
| Ig E (ME/ml) | 73.07 ± 2.26 | 60.15 ± 5.49 | >0.05 |
| [symbol] (conv. units.) | 11.07 ± 1.22 | 10.08 ± 0.83 | >0.05 |
| [symbol] (%) | 18.13 ± 0.92 | 20.08 ± 2.06 | >0.05 |
| [symbol] (%) | 40.1 ± 1.25 | 35.0 ± 3.7 | >0.05 |
| [symbol] | 2.24 ± 0.06 | 2.26 ± 0.07 | >0.05 |
| [symbol] (%) | 18.4 ± 0.36 | 19.0 ± 0.76 | >0.05 |
| [symbol] (%) | 38.5 ± 1.51 | 40.6 ± 1 [symbol] | >0.05 |
| [symbol] | 2.24 ± 0.1 | 2.16 ± 0.07 | >0.05 |

The exception is the serum immunoglobulin M class value, which showed a statistically significant increase in its concentration (0.63 to 0.08 g/L to 0.98 0.07 g/l).

During studies, an immune correcting effect of the medical preparation “RENESSANS” was installed under normal conditions. Maybe in terms of pathology, this effect will manifest itself more clearly.

To investigate the safety of the medical preparation “RENESSANS,” it was administered to animals intraperitoneally at various doses undiluted and diluted in a saline solution. The selection of the dose was based on recommendations for the maximum allowable amount of fluid for injection into the peritoneal cavity, which for rats is 5 ml. The choice of dose for injection was determined according to the classification of the toxicity of chemicals in accordance with ΓOCT (government standards) 12.1.007-76. The medical preparation was administered intraperitoneally once daily.

During the experiment, rats were divided into control and experimental groups. There were four experimental groups at different doses and timing of administration.

The medical preparation was administered intraperitoneally to animals with breeding in saline solution of sodium chloride, and undiluted.

1. The animals of the first group were injected intraperitoneally daily to 0.06 ml of the medical preparation 3.0 ml of physiological solution that exceeds recommended therapeutic dose 1.7 times. Rats were sacrificed after 5, 10 and 15 days after the beginning of experiment.
2. The animals of the second group were injected once by 0.66 ml of the medical preparation and 3.0 ml of physiological solution, which is more than the recommended therapeutic dose 11 times, the slaughter of animals was produced in 5 days after the start of administration.
3. The animals of the third group were injected once by 2 ml of preparation and 3.0 ml of saline solution, which is more than the recommended therapeutic dose 33 times and the slaughter of animals was produced within 10 days after the beginning of experiment.
4. The fourth group of animals was injected once by 5 ml of pure medical preparation without dilution, which exceeds the recommended therapeutic dose 83 times and the slaughter of animals was produced within 15 days after the beginning of the experiment.

Basic data of the analysis of the morphological composition of blood and leukocytic formula are shown in Table 3.

TABLE 3
Morphologic composition of blood of experimental animals

| | Control | | Series of experiments | | | |
|---|---|---|---|---|---|---|
| Blood index | pure | with NaCl | I | II | III | IV |
| Erythrocytes | 5.8 ± 0.1 | 6.3 ± 0.1 | 5.9 ± 0.1 | 6.1 ± 0.1 | 6.1 ± 0.1 | 6.2 ± 0.1 |
| 10⁹/l | | | | | | |
| Hbg/l | 116 ± 2 | 118 ± 2.5 | 105 ± 3.0 | 117 ± 3.0 | 114 ± 3.0 | 115 ± 3.0 |
| Color index | 0.9 ± 0.01 | 0.9 ± 0.01 | 0.85 ± 0.01 | 0.90 ± 0.01 | 0.90 ± 0.01 | 0.92 ± 0.01 |
| Segmented | 33 ± 1.5 | 32 ± 1.4 | 29 ± 2.0 | 31 ± 2.0 | 33 ± 2.0 | 32 ± 2.0 |
| neutrophils | | | | | | |
| 10⁹/l | | | | | | |
| Lymphocytes | 54 ± 1.2 | 60.5 ± 1.2 | 54 ± 1.0 | 58 ± 1.5 | 55 ± 2.0 | 58 ± 2.0 |
| 10⁹/l | | | | | | |
| Monocytes | 2 ± 0.1 | 2.5 ± 0.1 | 2.7 ± 0.2 | 4.3 ± 0.1 | 2.5 ± 0.2 | 3.0 ± 0.1 |
| 10⁹/l | | | | | | |
| Leukocytes | 9.2 ± 0.6 | 8.6 ± 0.8 | 8.5 ± 1.0 | 9.9 ± 1.0 | 8.9 ± 1.5 | 10.0 ± 2.0 |
| 10⁹/l | | | | | | |
| [symbol] | 1.6 ± 0.01 | 2 ± 0.01 | 1.3 ± 0.02 | 2.0 ± 0.07 | 2.5 ± 0.05 | 2.0 ± 0.01 |

As seen from Table 3, the study medication did not cause statistically significant changes in the blood cells of experimental animals, compared to control rats; therefore, the medicine has no toxic effects on the blood system. Histological examinations of animal organs in the control and experimental groups showed that the medical preparation “RENESSANS” did not cause changes in the histological structure of the internal organs of experimental animals, which also points to the non-toxicity of the investigational preparation.

Thus, the developed medical preparation “RENESSANS” has antibacterial, anti-ulcer and immune simulating effects at low toxicity and with the possibility of use in injection form.

What is claimed:

1. A medical preparation, which has anti-bacterial and anti-ulcer immune simulating actions containing iodine, potassium iodide, starch, glucose, ascorbic acid, sodium chloride and distilled water, comprising the following ratio of components (wt %):

| | |
|---|---|
| Iodine | 0.4-2.0 |
| Potassium Iodide | 0.8-4.0 |
| Starch | 10.0-40.0 |
| Ascorbic acid | 0.4-2.0 |
| Glucose | 1.2-4.8 |
| Sodium chloride | 0.3-1.8 |
| Purified water | all the rest |

2. The medical preparation of claim 1, characterized in that the medical preparation is intended for intravenous, intramuscular or oral administration to a patient in a pharmaceutically effective amount.

3. The medical preparation of claim 1, the use of the medical preparation being for treatment of viral and bacterial infections of mammals, including humans.

4. The medical preparation of claim 1, the use of the medical preparation being for treatment of peptic ulcer disease of mammals, including humans.

5. The medical preparation of claim 1, the use of the medical preparation being for increasing immunity of mammals, including humans.
